(19) United States
(12) Patent Application Publication
(10) Pub. No.: US 2022/0225917 A1
(43) Pub. Date: Jul. 21, 2022

(54) ELECTRONIC DEVICE, SYSTEM AND METHOD FOR PREDICTING THE PERFORMANCE OF AN INDIVIDUAL HUMAN DURING A VISUAL PERCEPTION TASK

(71) Applicants: TOYOTA MOTOR EUROPE, Brussels (BE); MIND VISION LABS, London (GB)

(72) Inventors: Jonas Ambeck-Madsen, Brussels (BE); Nilli Lavie, London (GB); Josef Schoenhammer, London (GB); Luke Palmer, London (GB)

(21) Appl. No.: 17/610,978

(22) PCT Filed: May 15, 2019
(86) PCT No.: PCT/EP2019/062433

§ 371 Date: Nov. 12, 2021

(51) Int. Cl. A61B 5/18; A61B 5/1455; A61B 5/00; B60W 40/08; B60W 50/00; G06V 20/56; G06V 10/40; G06V 10/764

(52) U.S. Cl. CPC A61B 5/18; A61B 5/14553; A61B 5/6893; B60W 40/08; B60W 50/0097; B60W 2420/40; G06V 10/40; G06V 10/764; A61B 5/7267; B60W 2540/221; G06V 20/56

(57) ABSTRACT

The invention relates to an electronic device (1) for predicting the visual perceptual task performance of an individual human. The electronic device is configured to: •receive an output of a first sensor device configured to measure the working memory load at the frontal cortex of the human, and •predict the visual perceptual task performance as a function of said sensor output. The invention further relates to a system and a method.

### CROSS-REFERENCE TO RELATED APPLICATIONS

This application is a National Phase of International Application No. PCT/EP2019/062433 filed May 15, 2019, the entire contents of which are herein incorporated by reference.

### FIELD OF THE DISCLOSURE

The present disclosure is related to an electronic device, system and method for predicting the visual perceptual task performance of an individual human, in particular the human's current task performance of perceiving a visual stimuli.

### BACKGROUND OF THE DISCLOSURE

The human's tasks performance of perceiving a visual stimuli, e.g. an suddenly occurring obstacle during a driving task, may vary due to different reasons. In particular, a task like e.g. driving often involves situations in which the driver's (i.e. the individual human's) mind may be disengaged from the driving task because the driver mind is intensely focused on other matters not related to the driving. For example, the driver may make some mental planning for a meeting at the destination of the drive. Critically, when the driver is intensely engaged in such mental activity this can impair his/her visual perception during driving, and produce a phenomenon of ‘looking but not seeing’.

It is thus desirable to predict the current (i.e. temporarily variable) visual perceptual task performance of the driver, i.e. an individual human, especially since there may be cases where there is no overt indication in the driver environment (both in and outside the car) that can indicate the driver's level of mental state of ‘mind off the road’ (and off the driving task). For example, the driven road may be almost empty and anyway the visual perceptual task performance of the driver is riskily low due to a ‘mind off the road’ state.

Different techniques have been proposed for predicting a human's mental engagement. For example, some methods assess mental engagement with electroencephalography (EEG) which measures changes in the electric field on the scalp that are related to changes in the electric field of patches of neurons in the brain. A considerable drawback of this method is that EEG signals are strongly distorted by other sources of electric field changes such as muscular activity, (e.g. from head movements, mouth movement, eye movements, blinks, and so forth) even when the active muscles are distant to the EEG sensors (e.g. arm movement).

Furthermore, it also has been proposed to measure pupil dilation, in order to assess mental engagement.

For example, Unsworth and Robison (2015) have related pupil size to an individual working memory capacity. They found that the increase in pupil size with the set size of a working memory task was bounded by an upper limit, reaching a plateau which is sensitive to the individual's working memory (WM) capacity: The pupil of individuals with lower capacity reach their maximum dilation at lower difficulty levels than those with higher capacity, cf.: Unsworth, N., & Robison, M. K. (2015). Individual differences in the allocation of attention to items in working memory: Evidence from pupillometry. Psychonomic Bulletin & Review, 22(3), 757-765.

Noah (2016) used pupil dilation to measure workload in a monitoring task and used enumeration of medium quantities to define participant profiles, cf.

Noah, B. (2016). Modeling Mental Workload with Eye Tracking Using Latent Profile Analysis.

With regard to applications of pupil dilatory responses, there exist methods for estimating fatigue levels and mental alertness from pupil responses to stimuli, cf. e.g. US20180220885A1 and US20060203197A1. Furthermore U.S. Pat. No. 6,090,051A and EP2798525A1 disclose to measure cognitive work-load from pupillary responses. Moreover technologies are known for estimating levels of cognitive load from various in-car sensors including the pupil, cf. e.g. US20180009442A1.

However, pupil dilation methods are highly susceptible to changes in ambient light intensity, and level of arousal.

Beside this, several studies found that higher levels of load on working memory (active maintenance of items in mind until a retrieval cue is presented) were associated with fNIRS results of higher levels of brain activity in medial or lateral prefrontal cortex. To generate increases in mental engagement, different working memory load tasks were used. In studies using ‘n-back’ tasks, participants had to monitor for repetition of identical items (e.g. digits or letters) interspersed among other items, and working memory load increased with the number of interspersed items, cf. e.g.:
Aghajani, H., Garbey, M., & Omurtag, A. (2017). Measuring Mental Workload with EEG+fNIRS. Frontiers in Human Neuroscience (11) 359, pp. 1-20, and Fairclough, S. H., Burns, C., & Kreplin, U. (2018). FNIRS activity in the prefrontal cortex and motivational intensity: impact of working memory load, financial reward, and correlation based signal improvement. Neurophotonics, 5 (3), pp. 1-10.
Durantin et al. (2014) used fNIRS to investigate the brain areas associated with working memory load during a ‘plane’ tracking task, cf.:
Durantin, G., Gagnon, J.-F., Tremblau, S. & Dehais, F. (2014). Using near infrared spectroscopy and heart rate variability to detect mental overload. Behavioural Brain Research 259, pp. 16-23.

Two recent studies used fNIRS to find out which brain areas are linked to drivers' working memory load from monitoring of events on the road while driving in a simulator (Unni et al., 2017; Scheunemann et al., 2019), cf.:
Unni, A., Ihme, K., Jipp, M. & Rieger, J. W. (2017). Assessing the Driver's Current Level of Working Memory Load with High Density Functional Near-infrared Spectroscopy: A Realistic Driving Simulator Study. Frontiers in Human Neuroscience (11) 167, pp. 1-14, and
Scheunemann, J., Unni, A., Ihme, K., Jipp, M. & Rieger, J. W. (2019). Demonstrating Brain-Level Interactions Between Visuospatial Attentional Demands and Working Memory Load While Driving Using Functional Near-Infrared Spectroscopy. Frontiers in Human Neuroscience (12) 542, pp. 1-17.

In both studies, working memory load was varied in an n-back task on the speed limit signs. When participants passed a speed sign they had to adapt their speed to the speed sign n signs ago. Working memory load increased with n, as indicated by increasing fNRIS activity.
Gateau et al. (2015) varied working memory load and measured related brain activity in dorsolateral prefrontal cortex) with fNIRS, while participants perform a flight in a flight simulator, cf.:
Gateau, T., Duratin, G. Lancelot, F., Scanella, S. & Dehais, F. (2015). Real-time State Estimation in a Flight Simulator using fNIRS. PLoS ONE (10) 3, pp. 1-19.

Finally, WO2017211395 (A1) relates to a control device, system and method for a vehicle for determining the perceptual load of a visual and dynamic driving scene, in particular of an uncontrolled, dynamically changing visual scene that the driver must perceive to carry out the driving task.

However, no known technology exists for predicting the visual perceptual task performance of an individual human in a reliable manner which is e.g. suitable to be applied as a predictor of the current driver performance and more particularly to predict ‘mind off the road’ states.

### SUMMARY OF THE DISCLOSURE

Currently, it remains desirable to provide an electronic device and method for predicting the visual perceptual task performance of an individual human, in particular in a reliable manner, e.g. for predicting whether a human's (or driver's) mind is disengaged from the (driving) task.

Therefore, according to the embodiments of the present disclosure, an electronic device for predicting the visual perceptual task performance of an individual human.

The electronic device is configured to:
receive an output of a first sensor device configured to measure the working memory load at the frontal cortex of the human,
predict the visual perceptual task performance as a function of said sensor output.

By providing such an electronic device, it becomes possible to reliably detect high levels of drivers' mental state of ‘mind off the road’ which involve a higher level of engagement in thought that is shown to impair their visual perception (specifically of motion). Importantly the proposed solution does not require any signals in the driving environment since it is based on measuring the driver brain activity in a non-intrusive manner.

The control device may be used as a basis for triggering warning signals during highly automated driving, e.g. when a ‘mind off the road’ state is predicted.

However, the present disclosure is not limited to the mentioned driving scenario. The electronic device of the present disclosure may also be used in context of other visual tasks which may be implied by e.g. controlling an aircraft or any other machine.

The present disclosure may specifically relate the higher level of mental engagement (higher ‘working memory’ load) as reflected in a brain activity pattern (changes in oxygenated and de-oxygenated haemoglobin concentration) measured with (e.g. fnirs) sensors (e.g. placed over lateral prefrontal cortex) which is shown to impair visual perception (specifically perception of motion).

Accordingly, the electronic device may predict, based on sensor data of e.g. a fNIRS sensor, human (e.g. driver) states of high mental engagement (i.e. ‘working memory load’) that lead to a cost to visual perception (specifically perception of motion) as required for e.g. driving. In particular, the electronic device allows to detect a higher level of brain activity in e.g. the lateral prefrontal cortex that is indicative of a higher level of mental engagement. This mental engagement results in a cost to visual perception. Importantly, the electronic device allows the detection of mental working memory load without any changes in the external environment during the higher mental activity.

Furthermore, in contrast to pupil dilation measurement, the present disclosure has the advantage that working memory load measurements, i.e. of brain activity measurements, (e.g. by a fNIRS sensor) is insensitive to the levels of light and thus superior to measures of pupil dilation. The measure of mental activity can be obtained without relying on any particular changes in eye movements. For example, a person may stare at one point during the driving with their mind being off the road at a high or low level of mental engagement (and conversely disengagement from perception) which can be detected by the electronic device according to the present disclosure. FNIRS sensors are also less sensitive to noisy signals due to fluctuations in arousal levels.

The first sensor device may comprise at least one functional near-infrared spectroscopy (fNIRS) sensor configured to be placeable in the human's head, specifically on the frontal part of the cortex.

For example, brain activity of a person watching moving objects may be measured with functional near infra-red spectroscopy (fNIRS). As an exemplary advantage, the pattern of fNIRS activity can clearly distinguish high mental load (more intense mental engagement, working memory load) that results in a cost to the perception of motion.

In contrast to known EEG measuring techniques, fNRIS measures of blood oxygenation levels are not sensitive to this type of muscular motion and changes of electrical field due to muscular activity.

Moreover, fNIRS measures local brain activity (where the optode channels are), which is not interfered by changes in blood oxygenation due to activity in other regions of the brain, measured in other channels (while EEG measures spatially diffuse activity, which can be influenced by irrelevant EEG signal generators of the brain). This makes the fNIRS an improved method to find specific brain activity patterns associated with high working memory load for which the regional-specific brain activity (e.g. in medial prefrontal cortex and in DLPFC) is well established.

The first sensor device may be configured to measure the working memory load only at the frontal cortex of the human.

Measuring the working memory load may comprise (or consist of) measuring a change of concentration levels of oxygenated (HbO2) and/or deoxygenated haemoglobin (HHb)) elicited by neuronal activation, e.g. in the underlying brain tissue at the frontal cortex of the human.

The electronic device may be configured to predict a decrease of the visual perceptual task performance, in case the measured working memory load increases.

The electronic device may be configured to receive data records representing a perceptual load of a visual and dynamic scene perceivable by the human.

The electronic device may be configured to predict the visual perceptual task performance additionally as a function of said data records.

The electronic device may be configured to predict a decrease of the visual perceptual task performance, in case the measured working memory load increases and at the same time the perceptual load does not increase.

Accordingly, the electronic device may additionally be complemented by or carry out a computer vision method (i.e. receive and process data records representing a perceptual load of a visual and dynamic scene perceivable by the human) that measures changes in load in the (driving) scene. For example, states of mind ‘off the road’ will result in a pattern of activity as will be described in detail below, and the computer vision method can confirm that pattern of activity is not correlated with changes in the level of perceptual load in the scene.

The present disclosure further relates to a system for predicting the visual perceptual task performance of an individual human. The system comprises:
a sensor system configured to produce data records of working memory load of at the frontal cortex of the human and/or of perceptual load values of a visual and dynamic scene perceivable by the human, and
an electronic device as described above.
   The sensor system may comprises:

The sensor system may comprise a first sensor device (e.g. including at least one fNIRS sensor) configured to measure the working memory load at the frontal cortex of the human, and/or a second sensor device configured to sense perceptual load values of a visual and dynamic scene perceivable by the human.

The second sensor device may comprise a scene sensor sensing the visual scene and may be configured to:

extract a set of scene features from the sensor output, the set of scene features representing static and/or dynamic information of the visual scene, and
determine the perceptual load of the set of extracted scene features based on a predetermined load model, wherein
the load model is predetermined based on reference video scenes each being labelled with a load value.

The second sensor device may in particular be a control device as described in WO2017211395 (A1).

Accordingly, the scene features may be extracted directly from the visual driving scene. Furthermore the perceptual load of the set of extracted scene features may be determined. By labelling reference video scenes with e.g. crowd sourced load values, i.e. by combining visual scene information with crowd-sourced load labels, the second sensor device may correctly learn, classify and identify the perceptual load in driving from the set of scene features extracted from a visual driving scene using a data-driven approach.

Furthermore, by providing such a second sensor device, the perceptual load may be determined based on a load model which is predetermined based on reference scenes or reference data each being labelled with a load value. Accordingly, the load model may be trained by reference video scenes with corresponding load values. The mapping between reference scenes and the respective load values, i.e. the labelling, may involve crowd sourcing, i.e. may be based on the evaluations of test persons. In other words, this mapping may be human based, in order to integrate information about the way humans experience perceptual load of the reference video scenes.

The reference video scenes desirably provide a set of exemplary visual driving scenes, e.g. a set of more than 1000 scenes, e.g. 1800.

Accordingly, it is possible that the load model and hence the second sensor device may learn the perceptual load of the reference scenes as related to the judgments of the crowd-sourced drivers (i.e. test persons). Based on this learnt information, the load model can be trained, in order to develop a general mapping function between a set of scene features (as an input of the mapping function) and resulting perceptual load (as an output of the mapping function). In other words, the load model becomes capable of determining the perceptual load of the visual driving scene, via its set of extracted scene features.

The determined perceptual load of the visual driving scene is desirably also expressed as a load value in the same format as the load values with which the reference video scenes have been labelled.

The load value may be expressed by one value, in particular a natural number, e.g. between 10 and 45, wherein for example 25 constitutes a mean perceptual load.

The visual and dynamic driving scene desirably corresponds to a driver's perspective. Hence, it desirably includes an outdoor visual driving scene, i.e. a scene of the environment of the vehicle, in particular in front of the vehicle (seen through the front window) and left and right to the vehicle (seen through the frontal side windows). It desirably further includes the driving mirrors. Moreover, it desirably includes the control panel of the vehicle, e.g. any screens and displays. In other words, it desirably includes all visual elements which influence the load of the driver related to the driving task.

The sensor may be an optical sensor, in particular at least one digital camera. The sensor is desirably oriented in the driving direction of the vehicle, in particular such that it senses the road in front of the vehicle. In addition, the sensor or further sensors may be oriented to the left and/or right side of the vehicle, in particular to sense the road left and/or right of the vehicle. Alternatively or additionally, also other sensor types may be used, e.g. radar (i.e. radio detection), x-ray and/or any acoustic (e.g. supersonic) sensors.

The sensor output may be a digital video or digital stream, in particular of a predetermined length (in the following also referred to as “video snippet”). A “sliding window” approach may be used to provide a continuous output of the perceptual load. Accordingly, a perceptual load value may be output for every frame of the video.

The disclosed second sensor device or the complete system may in particular be employed in driver support systems to indicate when the level of perceptual load on the road reaches a predetermined threshold (e.g. predetermined as a function of the predicted visual perceptual task performance of the individual human) that may require a warning signal to the driver to pay attention to the road.

Further, the second sensor device or the complete system may also be employed in the context of driver support systems, for example but not limited to the case of sudden brakes initiated by the driver support system. Also in such situations it is important for the automatic control system to be able to reliably determine the perceptual load of the driving scene.

The disclosed second sensor device or the complete system may also in particular be employed in the context of vehicle-to-driver interactions for highly automated vehicles, for example but not limited to the case of so-called take-over-requests, where the automatic control system requests a driver to re-take control over vehicle operation. In such situations it is important for the automatic control system to be able to reliably determine the perceptual load of the driving scene related to the driving task. A further exemplary case would be that the automatic control system takes over driving control, e.g. in case the system recognizes that the determined perceptual load exceeds a specific threshold.

The load model may comprise a mapping function between sets of scene features extracted from the reference video scenes and the load values.

Accordingly, as also explained above, the load model may be trained by mapping a set of scene features extracted from a reference video scene to a corresponding load value. Since this mapping may form a general regression/mapping function, the load model becomes capable of determining the perceptual load of any sensed visual driving scene, i.e. of its set of extracted scene features.

The load model may be configured to map a set of scene features to a perceptual load value.

Hence, as also explained above, the load model can map a set of scene features extracted from any sensed visual driving scene to a perceptual load value. Accordingly, the perceptual load of said driving scene can be determined.

The load model may be a regression model or a classification model between the sets of scene features extracted from the reference video scenes and the load values. In case of a classification model, it may be useful to additionally create load categories from the load values, e.g. get a model to classify high vs low load traffic scenes.

The determination of the load values of the reference video scenes may be human based, in particular based on crowdsourcing. Accordingly, the load values may be evaluated directly by humans (i.e. test persons).

For example, the determination of the load values may be based on a pairwise ranking procedure, i.e. on an algorithm which estimates ratings from pairwise comparisons, in particular based on the TrueSkill algorithm.

Accordingly, a known algorithm as e.g. the TrueSkill algorithm may be applied, in order to rank the reference video scenes with regard to their perceptual load. In order to do so, test persons may evaluate pairs of reference video scenes, in order to decide which of the two reference video scenes has the higher perceptual load. By presenting a multitude of different pairs to a plurality of test persons, an overall ranking between all reference video scenes can be determined. This overall ranking may be expressed as the load values, with which the reference video scenes have been labelled. In other words, the overall ranking may be expressed as the load values which are then assigned to the reference video scenes.
The TrueSkill algorithm is also described in Herbrich, R., Minka, T., and Graepel, T. (2006): “Trueskill: A bayesian skill rating system”, Advances in Neural Information Processing Systems, pages 569-576.
Instead of the TrueSkill algorithm, also the Elo model (Elo, A. (1978): “The Rating of Chessplayers, Past and Present”, Arco. ISBN 0-668-04721-6), the Glicko system (Glickman, Mark E., (1999): “Parameter estimation in large dynamic paired comparison experiments”, Applied Statistics, 48, 377-394), or the BTL (Bradley Terry Luce) algorithm for converting pairwise comparisons to ratings may be applied.

Instead of a pairwise ranking procedure, also another number of reference video scenes may be compared in the ranking procedure, e.g. a triplet, four, or more reference video scenes.

It is also possible that the second sensor device and/or the electronic device is configured to continuously train the load model by monitoring the driver during the driving scene, in particular the driver's responses to the visual scene and/or acoustic signals emitted by the vehicle. Accordingly, the second sensor device may further optimize the load model “on the go”, i.e. while the vehicle is driven. For this purpose, the driver may be monitored, e.g. by one or more cameras, etc., in order to measure the physiological response (e.g. pupil dilation) of the driver during driving. In particular, the driver's responses to acoustic signals emitted by the vehicle may be measured. Further, additional response time and the response behavior, including the driving behavior, such as e.g. sudden braking, steering, etc. may be monitored in conjunction.

A monitored behavior of the driver during the driving scene not matching the determined perceptual load may serve to on-line up-date said mapping function. Accordingly, based on the monitored information regarding the behavior of the driver during the driving scene it may be judged, whether the determined load appears to be correct or not, and the load model may be optimized based on the judgement. For example, in case the determined load value indicates a low perceptual load of the driving scene, but the driver's behavior suggests a high perceptual load (e.g. due to a low pupil response and a hectic reaction like sudden braking, steering, etc.), the load model may be adapted accordingly. Hence, any situations not matching previous results of the mapping function (i.e. the load model) may serve to on-line up-date said mapping function.

Furthermore, it is also possible that any driving scenes that already have been monitored by the sensor may be used as further reference video scenes, with which the load model may be trained.

The set of scene features may comprise a range of spatio-temporal features, the set of scene features being in particular described in vector form.

The set of scene features may comprise improved dense trajectory (iDT) features and/or a 3-dimensional convolutional neural network (C3D) features.
Improved dense trajectory (iDT) features are also described in Wang, H. and Schmid, C. (2013): “Action recognition with improved trajectories”, IEEE International Conference on Computer Vision, Sydney, Australia.
Convolutional 3D (C3D) features are also described in Tran, D., Bourdev, L., Fergus, R., Torresani, L., and Paluri, M. (2015): “Learning spatiotemporal features with 3d convolutional networks”, IEEE International Conference on Computer Vision, pages 4489-4497.

The load model may be a linear regression model, a kernel regression model, a support vector regression model, a ridge regression model, a lasso regression model, or a random forest regression model. The load model may be in particular a multi-channel non-linear kernel regression model.

The load model may be a linear regression model, wherein the set of scene features (in particular of the sensed driving scene) being an input scene feature vector x is mapped to the perceptual load being an output perceptual load value y=f(x) through a linear mapping function f(x)=w^{T}x+b=w₁*x₁+w₂*x₂+w₃*x₃ . . . +b, the function being a weighted sum of the input dimension values of the feature vector x, wherein weighted parameters w are assigned to each dimension value in the feature vector x and a bias term b centres the output at a particular value.

Alternatively the load model may be a multi-channel non-linear kernel regression model, where the mapping function is f(x)=w^{T} φD(x)+b, wherein φ(x) is a transformation function of the input feature vectors to a non-linear kernel space.

The disclosure further relates to a vehicle comprising an electronic device or a system as described above.

Accordingly, also a plurality of sensors may be used, in order to sense (i.e. perceive) the driving scene. For example, two sensors might be used, in order to obtain three dimensional information of the driving scene, as well as surround view type sensor configuration, and any combination hereof. Furthermore, a cabin sensor may be used, e.g. an IR or RGB camera, in order to measure the pupil diameter of the driver.

Finally, the present disclosure relates to a method of predicting the visual perceptual task performance of an individual human, comprising the steps of:
receiving an output of a first sensor device configured to measure the working memory load at the frontal cortex of the human, and
predicting the visual perceptual task performance as a function of said sensor output.

The method may comprise further steps corresponding the characteristics of the electronic device, as described above.

It is intended that combinations of the above-described elements and those within the specification may be made, except where otherwise contradictory.

It is to be understood that both the foregoing general description and the following detailed description are exemplary and explanatory only and are not restrictive of the disclosure, as claimed.

The accompanying drawings, which are incorporated in and constitute a part of this specification, illustrate embodiments of the disclosure and together with the description, and serve to explain the principles thereof.

### BRIEF DESCRIPTION OF THE DRAWINGS

FIG. 1 shows a block diagram of a system with an electronic device according to embodiments of the present disclosure;

FIG. 2 shows a schematic flow chart illustrating an exemplary method of determining the perceptual load according to embodiments of the present disclosure;

FIG. 3 shows a flow chart illustrating the exemplary method of FIG. 2 in more detail;

FIG. 4 shows a schematic diagram of dense trajectory extraction of a visual scene by dense trajectories;

FIG. 5 shows a diagram illustrating the C3D system architecture according to embodiments of the present disclosure;

FIG. 6 shows a schematic diagram illustrating the training of the load model according to embodiments of the present disclosure; and

FIG. 7 shows an example of the labelling procedure to compare a pair of reference video scenes, which is subsequently fed into the TrueSkill algorithm.

### DESCRIPTION OF THE EMBODIMENTS

Reference will now be made in detail to exemplary embodiments of the disclosure, examples of which are illustrated in the accompanying drawings. Wherever possible, the same reference numbers will be used throughout the drawings to refer to the same or like parts.

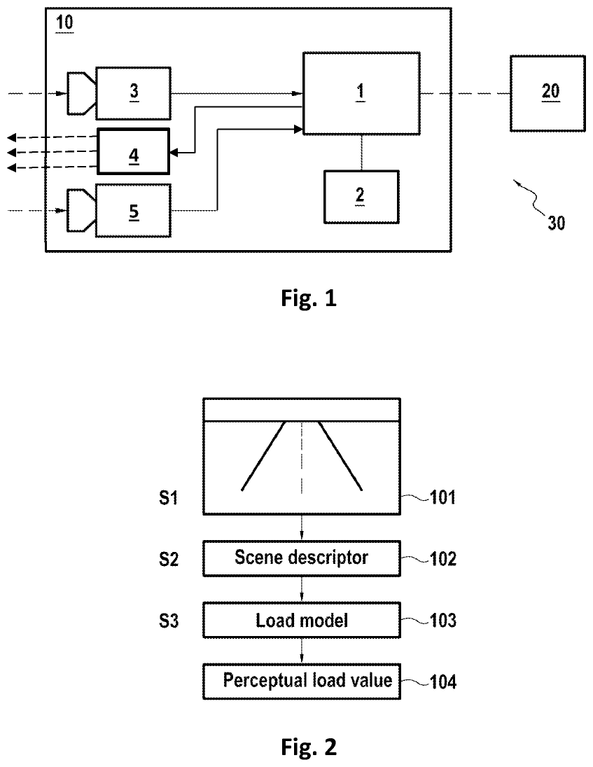

FIG. 1 shows a block diagram of a system 30 with an electronic device 1 according to embodiments of the present disclosure. In one example, the system 30 may be a vehicle 10. In another example, the system 30 is a test system 10 configured to predict the visual perceptual task performance of an individual human, i.e. a test person.

The electronic device 1 is connected to or comprises a data storage 2. Said data storage may be used to store data records recorded by a first sensor device (e.g. one or several fNIRS sensors) and data records of perceptual load values of a visual and dynamic scene perceived by a human, e.g. the driver. It may additionally store e.g. a load model. As described in the following, said load model maybe used to determine the perceptual load of the visual and dynamic scene.

The electronic device 1 may additionally carry out further functions in the system 30, e.g. in the vehicle 10. For example, the electronic device may also act as the general purpose ECU (electronic control unit) of the vehicle.

The electronic device 1 may comprise an electronic circuit, a processor (shared, dedicated, or group), a combinational logic circuit, a memory that executes one or more software programs, and/or other suitable components that provide the described functionality. In the example of a vehicle 10, the sensor 5 may be installed in the vehicle cabin, in order to measure the working memory load of the driver. In the example of the test system 10, it measures the working memory load of the test person.

The electronic device 1 may be connected to a sensor 5 in particular including at least one fNIRS sensor. The sensor 5 is configured to measure the working memory load, e.g. at the frontal cortex of a human. For example a plurality of fNIRS sensors may be placed on the head of a person, specifically on the frontal part of the cortex. Using a 3D computer model of the head, the relevant brain areas may be projected to locations on the head surface. These locations can then be found on the scalp by their relative distance to anatomical landmarks (i.e., nasion, left and right pre-auricular points, and innion). To validate the positioning of the fNIRS sensors on the person's head, the positions may be measured with a 3D digitizer, and projected on the brain in the 3D computer model.

The fNIRS signal may be recorded by the electronic device. To obtain the fNIRS signal changes related to neuronal processing of the working memory task, the signal may be filtered in several steps to remove low frequency changes (e.g. due to any slow movement of the fNRIS sensors on the scalp), mid-level frequency changes (due to heart rate activity and respiration), and/or high frequency changes (due to sudden movements of the fNIRS sensors on the scalp). The signal may then be analysed by a general linear model (GLM) that fits a model of the fNIRS signal changes related to the working memory task to the actual data, separately for each level of working memory load. The parameters estimated in the GLM may then be analysed by the electronic device 1.

The electronic device 1 may be further connected to an optical sensor 3 in particular a digital camera 3, at least in the example of the vehicle 10. The electronic device 1 and the digital camera may be comprised by a vehicle 10. The digital camera 3 is configured such that it can record a visual driving scene of the vehicle 10. The digital camera is desirably oriented in the driving direction of the vehicle, i.e. such that it records in particular the road in front of the vehicle. It is also possible to use several cameras 3. Accordingly, it may also be reasonable to use several sensors (e.g. cameras), in order to cover the complete field of view of the driver.

The output of the sensor 5 and/or the optical sensor 3, in particular a recorded video stream, is transmitted to the electronic device 1. Desirably, the output is transmitted instantaneously, i.e. in real time or in quasi real time. Hence, the measured working memory load and/or perceptual load of the recorded driving scene can also be determined by the electronic device in real time or in quasi real time.

The optical sensor 3 alone or the combination of optical sensor 3 and electronic device 1 may also form a second sensor device according to the present disclosure, i.e. a control device as described in WO2017211395 (A1).

In case of the example of the test system 10, the system may comprise a task generator 4 controllable by the electronic device 1, in particular instead of the optical sensor 3. The task generator 4 may be in one example a display indicating a predetermined task to be performed by the test person. Since in this case the task is predetermined, the perceptual load of the task, as it is perceivable by the test person, is known to the electronic device 1. For example, a motion perception task is presented which may comprise detecting the direction of motion that occurred for a short period, from among randomly moving field of dots. The findings show that the motion direction perception threshold (the minimum proportion of dots moving in the same direction) is higher under high than low working memory load, indicating that higher working memory load (which takes the test person's mind off the visual perception task), as detected with increased activity in lateral frontal brain regions (shown with the fNIRS) impaired motion perception.

The system 30 may comprise additionally a server 20. The server 20 is used to train and eventually update the load model. For this purpose, the electronic device 1 may be connectable to the server. For example the electronic device 1 may be connected to the server 20 via a wireless connection. Alternatively or additionally the electronic device 1 may be connectable to the server 20 via a fixed connection, e.g. via a cable.

FIG. 2 shows a schematic flow chart illustrating an exemplary method of determining the perceptual load according to embodiments of the present disclosure. The method may be carried out by the electronic device 1. The method comprises two steps: In the first step (step S2), a set of scene features is extracted from the video. In the second step (step S3), the load model providing a mapping function is applied. In other words, a mapping function between the sets of scene features and perceptual load values is applied.

In more detail, it is at first provided a record of a visual driving scene in step S1. As described above, the visual driving scene is recorded by a sensor, in particular a digital camera. From the output of the sensor (e.g. a video stream) fixed duration video snippets 101 (e.g. 2 second long clips) are taken. Hence, the video snippets may be processed in the method of FIG. 2 consecutively.

In step S2 a set of scene features 102 (also referred to as a scene descriptor) is extracted from the current video snippet 101. As described in more detail in the following, the set of scene features may be expressed by a feature vector.

In step S3 the set of scene features 102 is passed through the load model 103, which may be a regression model learnt from crowdsourcing. As a result a perceptual load value 104 indicating the perceptual load of the video snippet 102 is obtained.

The method of FIG. 2 may be repeated for every single video snippet.

The method of FIG. 2 may be may be obtained using different regression models.

The determination of the perceptual load may also be regarded as an estimation, as it is not necessarily completely precise.

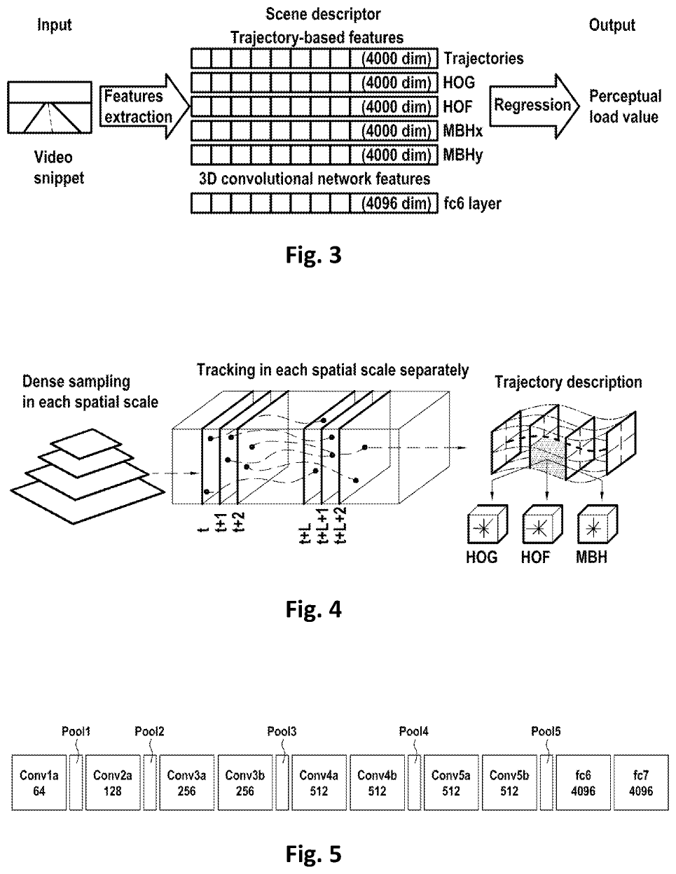

FIG. 3 shows a flow chart illustrating the exemplary method of FIG. 2 in more detail. In particular, the set of extracted scene features is shown in more detail, as it will be described in more detail in the following.

The goal of scene feature extraction is to describe the content of a video in a fixed-length numerical form. A set of scene features may also be called a feature vector. The visual information of the driving scene contributes to determine the perceptual load by extracting appearance and motion features of the visual driving scene. In order to extract the visual information, improved dense trajectory (IDT) features and 3D convolutional (C3D) features are desirably extracted from the video snippet, as it is described below. Such features constituting a set of scene features are then passed through the load model, which may be a regression model, in order to calculate a perceptual load value indicating the perceptual load of the video snippet.

Improved Dense Trajectories (IDT)

In improved dense trajectories, videos are represented as visual features extracted around trajectories of primitive interest points. Trajectories are the tracked (x,y) image location of “interest points” over time. Such “interest points” may be parts of an image which are salient or distinct, such as corners of objects. The interest points may be detected using the SURF (“Speeded Up Robust Features”) algorithm and may be tracked by median filtering in a dense optical flow field of the video.

FIG. 4 shows a schematic diagram of dense trajectory extraction of a visual scene by dense trajectories. As shown, dense trajectories are extracted for multiple spatial scales, e.g. 4 to 8 spatial scales, and then local features are computed within a space-time volume around the trajectory. Such an action recognition by dense trajectories is also described in Wang, H. and Schmid, C. (2013): “Action recognition with improved trajectories”, IEEE International Conference on Computer Vision, Sydney, Australia, which disclosure is incorporated herein in its entirety. Spatial scales is commonly refers to the sampling for the trajectories. It means that the trajectories are sampled across the image with different numbers of pixels in between them. For example, at scale 1 there is a spacing of 5 pixels, at scale 2 there is a spacing of 10 pixels etc.

Histograms of Oriented Gradients (HOG), Histograms of Optical Flow (HOF), and Motion Bounded Histograms (MBH) features in the x- and y-directions are extracted around each trajectory, in addition to the Trajectory features themselves (i.e. the normalized x,y location of each trajectory).

A Bag of Words representation is desirably used to encode the features. In the Bag of Words representation, a 4000-length dictionary of each trajectory feature type (Trajectory, HOG, HOF, MBHx, MBHy) is learnt. That is, every possible feature type is quantized into a fixed vocabulary of 4000 visual words, and a video is then encoded as a histogram of the frequency of each type of visual word. This results in a 20,000 dimensional feature vector (i.e. 5×4000-length feature vectors).

Convolutional 3D (C3D) Features

Convolutional 3D (C3D) features are a type of “deep neural network” learnt feature where features are automatically learnt from labelled data. A hierarchy of video filters are learnt which capture local appearance and motion information. A C3D network for feature extraction must first be trained before it can be used. A pre-trained network can be used (i.e. it has been trained on other data, and learns to extract generic video descriptors). For example the pre-trained model may be trained from a set of a million sports videos to classify sports. This learns generic motion/appearance features which can be used in any video regression/classification task. Alternatively or additionally for the training the labelled reference videos may be used, in order to fine-tune a C3D network.

FIG. 5 shows a diagram illustrating the C3D system architecture according to embodiments of the present disclosure. In the diagram ‘Cony’ represents a layer of convolutional video filters; ‘Pool’ represents max-pooling which subsamples the convolution output; and ‘ FC’ represents a fully connected layer which maps weighted combinations of features to output values. The final set of scene features comprises 4096 dimensions and represents a weighted combination of video filters that represents the motion and appearance of the video snippet. Convolutional 3D (C3D) features are also described in Tran, D., Bourdev, L., Fergus, R., Torresani, L., and Paluri, M. (2015): “Learning spatiotemporal features with 3d convolutional networks”, IEEE International Conference on Computer Vision, pages 4489-4497, which disclosure is incorporated herein in its entirety.

Training the Load Model

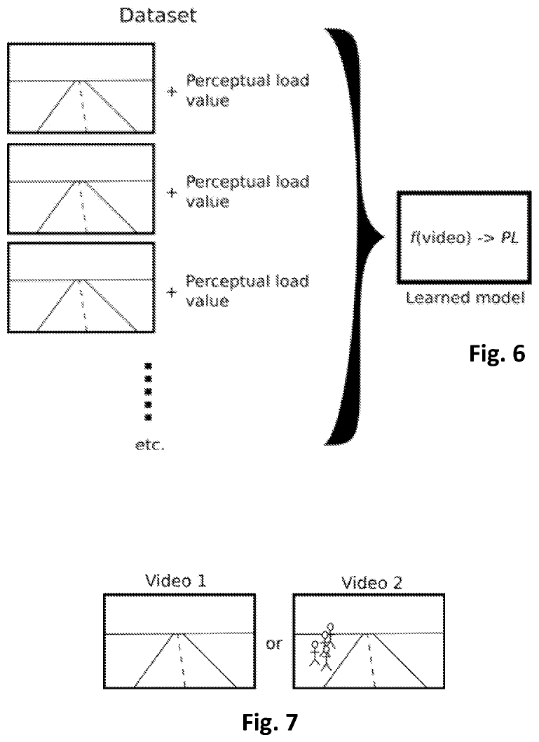

FIG. 6 shows a schematic diagram illustrating the training of the load model according to embodiments of the present disclosure. The load model is desirably a regression model. To train the regression model, examples of various driving scenarios, i.e. in particular the reference video scenes, and their corresponding load values are required so that the machine-learning algorithm can learn a mapping function from sets of scene features to perceptual load values.

So called “ground-truth” perceptual load values may be acquired through crowd-sourcing, where test persons, e.g. experienced drivers, watch and compare clips of driving footage in a pairwise-comparison regime which are then converted to video ratings. Pairwise comparisons provide a reliable method of rating items (compared to people assigning their own subjective load value which would provide inconsistent labels). Desirably a system is used where experienced drivers would label the relative perceptual load of videos and select which video from a pair is more demanding on attention to maintain safe driving. The collection of pairwise comparisons is desirably converted to ratings for each video using the TrueSkill algorithm.

An alternative method could be a done by a driver and a passenger who manually tag live streams by load value (a level of 1 to 5 for example) while driving for a long distance. During this test, also the load model might be trained. Accordingly, the live streams may be used as reference video scene, with which the load model is trained.

FIG. 7 shows an example of the labelling procedure to compare a pair of reference video scenes, which is subsequently fed into the TrueSkill algorithm.

The TrueSkill model assumes that each video has an underlying true load value. The probability of one video being ranked as higher load than another is based on the difference in their load values. After each comparison between a pair of videos, the video load values are updated based on which video was labeled as having higher load and their prior load value. All videos start off as having equal load values, and are updated after each comparison. The videos are compared until their corresponding load values no longer change. The final result is a load value for each video. The TrueSkill algorithm is also described in Herbrich, R., Minka, T., and Graepel, T. (2006): “Trueskill: A Bayesian skill rating system”, Advances in Neural Information Processing Systems, pages 569-576, which disclosure is incorporated herein in its entirety.

In the following the development of the load model being a regression model is described. Regression takes a fixed length feature vector (i.e. a set of scene features) and learns a mapping function to transform this to a single continuous output value (i.e. the labelled perceptual load of the reference video). The regression function is learnt from labelled training examples of input (i.e. the feature vector) and output (i.e. the labelled perceptual load values) pairs, and finds the function that best fits the training data.

Various types of regression models can be used, e.g. linear regression, kernel regression, support vector regression, ridge regression, lasso regression, random forest regression etc.

In the simplest case of linear regression, the input scene feature vector x, which is effectively a list of numbers {x₁, x₂, x₃, . . . , x_{N}}, is mapped to the output y (in our case the perceptual load value) through a linear function y=f(x), where the function is a weighted sum of the input numbers:

f(x)=w^{T}x+b−that is f(x)=w₁*x₁+w₂*x₂+w₃*x₃ . . . +b.

This is equivalent to fitting a line of best fit to the input data points, and will learn the parameters w (these are simply weights assigned to each feature/value/number in the feature vector, x) and a bias term b, which centers the output at a particular value.

In a better performing model, multi-channel non-linear kernel regression is used. This extends linear regression to cover complex non-linear relationships between input sets of scene-features and output load values through using a “kernel”. This is a transformation of the input feature vectors to a space where they can be better separated or mapped. The mapping function becomes:

f(x)=w^{T}φ(x)+b.

Then, regression is run in the combined kernel space. This is similar to fitting a line to 2D points, but in high dimensional space: a machine-learning algorithm finds the collection of weights, w, which minimizes the error in the perceptual load estimate on a ‘training-set’ (i.e. a subset of the whole dataset, in this case two thirds of the ˜2000 video-load value pairs). This optimal set of weights therefore defines the mapping that best transforms the set of scene features to a single value indicating the perceptual load.

In this way the load model comprising the regression function can be trained based on the training examples. Once the regression function is learnt, the same procedure may be run, when the electronic device 1 is used in the vehicle. Accordingly, in use of the electronic device 1, an input scene descriptor (i.e. a set of scene features) is extracted from a visual driving scene, and the regression function is applied on the input scene descriptor (i.e. the set of scene features), in order to calculate the output load value.

After learning the model, any video can be inserted and a perceptual load value will be output for every 2-second segment. A “sliding window” approach is used to provide a continuous output the perceptual load value (i.e. a value can be output for every frame of the video). Of course, the segment may also be short or longer than 2 seconds.

Throughout the description, including the claims, the term “comprising a” should be understood as being synonymous with “comprising at least one” unless otherwise stated. In addition, any range set forth in the description, including the claims should be understood as including its end value(s) unless otherwise stated. Specific values for described elements should be understood to be within accepted manufacturing or industry tolerances known to one of skill in the art, and any use of the terms “substantially” and/or “approximately” and/or “generally” should be understood to mean falling within such accepted tolerances.

Although the present disclosure herein has been described with reference to particular embodiments, it is to be understood that these embodiments are merely illustrative of the principles and applications of the present disclosure.

It is intended that the specification and examples be considered as exemplary only, with a true scope of the disclosure being indicated by the following claims.

1. An electronic device predicting the visual perceptual task performance of an individual human, configured to:
receive an output of a first sensor device configured to measure the working memory load at the frontal cortex of the human,
predict the visual perceptual task performance as a function of said sensor output.

2. The electronic device according to claim 1, wherein
the first sensor device comprises at least one functional near-infrared spectroscopy (fNIRS) sensor configured to be placeable in the human's head, specifically on the frontal part of the cortex.

3. The electronic device according to claim 1, wherein
measuring the working memory load comprises measuring a change of concentration levels of oxygenated (HbO2) and/or deoxygenated haemoglobin (HHb) elicited by neuronal activation in the underlying brain tissue at the frontal cortex of the human.

4. The electronic device according to claim 1, configured to predict a decrease of the visual perceptual task performance, in case the measured working memory load increases.

5. The electronic device according to claim 1, configured to receive data records representing a perceptual load of a visual and dynamic scene perceivable by the human, and predict the visual perceptual task performance additionally as a function of said data records.

6. The electronic device according to claim 5, configured to predict a decrease of the visual perceptual task performance, in case the measured working memory load increases and at the same time the perceptual load does not increase.

7. A system for predicting the visual perceptual task performance of an individual human, comprising:
a sensor system configured to produce data records of working memory load of at the frontal cortex of the human and/or of perceptual load values of a visual and dynamic scene perceivable by the human, and
an electronic device according to claim 1.

8. The system according to claim 7, wherein the sensor system comprises:
a first sensor device configured to measure the working memory load at the frontal cortex of the human, and/or
a second sensor device configured to sense the perceptual load of a visual and dynamic scene perceivable by the human.

9. The system according to claim 7, wherein
the second sensor device comprises a scene sensor sensing the visual scene and is configured to:
extract a set of scene features from the sensor output, the set of scene features representing static and/or dynamic information of the visual scene, and
determine the perceptual load of the set of extracted scene features based on a predetermined load model, wherein
the load model is predetermined based on reference video scenes each being labelled with a load value.

10. The system according to claim 7, wherein
the load model comprises a mapping function between sets of scene features extracted from the reference video scenes and the load values.

11. The system according to claim 7, wherein
the load model is at least one of a regression model and a classification model between the sets of scene features extracted from the reference video scenes and the load values.

12. A vehicle comprising:
an electronic device according to claim 1.

13. A method of predicting the visual perceptual task performance of an individual human, comprising the steps of:
receiving an output of a first sensor device configured to measure the working memory load at the frontal cortex of the human, and
predicting the visual perceptual task performance as a function of said sensor output.

14. The system according to claim 7, wherein
the load model is configured to map a set of scene features to a perceptual load value.

15. A vehicle comprising:
a system according to claim 7.
